(19) United States
(12) Patent Application Publication
(10) Pub. No.: US 2005/0170028 A1
(43) Pub. Date: Aug. 4, 2005

(54) Polysaccharide extract from Lycium barbarum as neuroprotective agent against beta-amyloid peptide neurotoxicity

(76) Inventors: Raymond Chuen-Chung Chang, Hong Kong (CN); Wai-Hung Yuen, Hong Kong (CN); Kwok-Fai So, Hong Kong (CN); S.Y. Zee, Hong Kong (CN)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 11/048,868

(22) Filed: Feb. 3, 2005

Related U.S. Application Data
(60) Provisional application No. 60/541,235, filed on Feb. 4, 2004.

(51) Int. Cl. A61K 31/715; C08B 37/00; A61K 35/78

(52) U.S. Cl. 424/777; 514/54; 536/123

(57) ABSTRACT

Extracts of Lycium barbarum serve as a neuroprotective agent against β-amyloid peptide neurotoxicity which thus permits their use for the treatment of Alzheimer's disease (AD) and for the prevention of neuronal loss in aging against the accumulation of β-amyloid peptide in the brain. Stress kinases (c-Jun N-terminal kinase and double-stranded RNA-dependent protein kinase) are used as a technological platform for screening neuroprotective drugs.

This disclosure is entitled to the benefit of U.S. provisional application Ser. No. 60/541,235, filed Feb. 4, 2004.

### FIELD OF THE INVENTION

The present invention relates to novel compositions from the extracts of the Lycium barbarum as a neuroprotective agent against β-amyloid peptide neurotoxicity which thus permits their use for the treatment of Alzheimer's disease (AD) and for the prevention of neuronal loss in aging against the accumulation of β-amyloid peptide in the brain. In another aspect, the present invention relates to a method for obtaining compositions having anti-β-amyloid peptide neurotoxicity from Lycium barbarum, and formulations containing said compositions. In a further aspect, the present invention relates to methods of using stress kinases (c-Jun N-terminal kinase (JNK) and double-stranded RNA-dependent protein kinase (PKR)) as a technological platform for screening neuroprotective drugs in all kinds of natural and synthetic chemicals or formulations against β-amyloid peptides neurotoxicity.

### BACKGROUND OF THE INVENTION

Alzheimer's disease is an age-related chronic neurodegenerative disease. The major symptoms of Alzheimer's disease are cognitive and language impairment (Yankner, 1996; Ray et al., 1998; Bossy-Wetzel et al., 2004). As it is a neurodegenerative disease, all coordination of patient movement becomes a problem. It has been predicted that over 14 million U.S. residents will suffer from this devastating disease (according to American Health Assistant Foundation). As aging populations rapidly increase in the next few decades, the occurrence of this disease will certainly be a major health problem and a burden on all governments in the world.

A major problem associated with Alzheimer's disease is neuronal loss. The death processes for neurons can be mediated via neuronal apoptosis, granulovacuolar degeneration or synaptosis (Stadelmann et al., 1999; Engidawork et al., 2001; Su et al., 2001; Chang et al., 2002a; Leroy et al., 2002; Scheff and Price, 2003; Yu et al., 2004). Neuronal apoptosis is mediated by different highly regulated biological processes. Activation of pro-apoptotic mechanisms have been found in the above three kinds of death processes. For examples, activation of caspases-3 and -8 have been found in postmortem human AD brain (Rohn et al., 2001; Su et al., 2001; Scheff and Price, 2003). Our laboratory at the University of Hong Kong and other laboratories have also found activation of caspase-3 and a pro-apoptotic stress kinase, the double-stranded RNA-dependent protein kinase (PKR), in granulovacuolar degenerative neurons in postmortem human AD brain sections (Chang et al., 2002a). Therefore, combating the death processes for neurons against aging, biological stress, or environmental toxins is major task in developing treatments of Alzheimer's disease.

The pathological features of Alzheimer's disease are the appearance of senile plaques and neurofibillary tangles. Accumulation or so-called deposit of insoluble β-amyloid (Aβ) peptide is the major component in such senile plaques (Ray et al., 1998). Therefore, it has been generally considered that increased production of insoluble Aβ peptide contribute to the formation of senile plaques. Indeed, it has been reported that production of insoluble Aβ peptide increases with age. High levels of Aβ peptide are toxic to neurons leading to neuronal apoptosis (Iversen et al., 1995; Saido, 2003; Bossy-Wetzel et al., 2004). Increased Aβ production in mutated human amyloid precursor protein (APP, K670N/M671L and V717F mutation) transgenic mice, and injection of Aβ into aged rhesus monkey or primate also show Alzheimer-type neuropathology, neuronal death and is correlated to memory deficits. Apart from in vivo animal studies, Aβ peptide has also been shown to exert neurotoxic effects leading to neuronal apoptosis in vitro in several laboratories. Therefore, Aβ peptide has long been regarded as a major toxin in the pathogenesis of Alzheimer's disease.

Apart from senile plaques formation, another pathological feature is neurofibillary tangles which has been considered to be related to the abnormal phosphorylation of microtubules-associated tau protein (Mandelkow and Mandelkow, 1998). Many environmental toxins, including Aβ peptide as well as glutamate, can trigger hyper-phosphorylation of tau protein (Sindou et al., 1994; Rapoport et al., 2002). Abnormal phosphorylation of tau protein can lead to derangement of the cytoskeleton and further trigger a cascade of neuronal apoptosis (Billingsley and Kincaid, 1997). Therefore, it has been considered that abnormal tau protein phosphorylation is the major factor contributing to the degeneration of neurons. However, controversial results have shown that abnormal tau phosphorylation can also occur in other types of neurodegenerative diseases without the problem of dementia. In addition, Aβ peptide neurotoxicity can trigger hyper-phosphorylation of tau. Therefore, it is expected that high levels of Aβ peptides would lead to cascades of pro-apoptotic pathways including the abnormal phosphorylation of tau protein.

Since the major symptom in Alzheimer's disease is cognitive impairment, nearly all, if not 100%, of the current treatments for Alzheimer's disease are based on targeting anti-cholinesterase. Up to third generation anti-cholinesterase drugs have been employed in the clinic. However, most of these drugs have side effects that may be intolerable for the patients. In addition, these drugs slow down the cognitive impairment but are not intended to safeguard neurons. If neurons are undergoing degeneration, problem of cognitive impairment will still occur. Furthermore, in view of the increasing population of aged people in the world, it is better to protect neurons far before their commitment into the initiation of apoptosis. Therefore, neuroprotection will increasingly become a useful and effective therapeutic strategy for the prevention of neuronal death in Alzheimer's disease and aging.

To prevent neuronal apoptosis, it is important to elucidate the molecular mechanisms of neuronal apoptosis. Our and other laboratories have demonstrated that the significance of stress kinases such as c-Jun N-terminal kinase (JNK) as well as double-stranded RNA-dependent protein kinase (PKR) in Aβ peptide neurotoxicity and in the pathogenesis of Alzheimer's disease (Chang et al., 2002b; Peel and Bredesen, 2003; Suen et al., 2003; Onuki et al., 2004). Cultured neurons exposed to Aβ peptide result in a rapid increase in the phosphorylation of JNK and PKR. Human postmortem Alzheimer's disease brain sections also display high immunoreactivity of phosphorylated PKR and JNK (Zhu et al., 2001; Chang et al., 2002a; Savage et al., 2002; Peel and Bredesen, 2003; Onuki et al., 2004). Therefore, we have recognized that examination of their phosphorylation in neurons exposed to Aβ peptide will be two key markers for neurons commiting into apoptosis. In this connection, examination of their phosphorylation in order to investigate whether drugs from, for instance, Chinese or herbal medicine can exert neuroprotection against Aβ peptide will be a useful technological platform for drug screening.

To promote longevity and prevention of degeneration, several strategies including physical exercise and caloric restriction should develop simultaneously. Daily consumption of anti-aging supplements like tonic Chinese medicine is a good strategy to reduce the susceptibility of cells to environmental toxins. Among various Chinese and oriental medicine, the dry fruit of Lycium barbarum (also known as Fructus Lycii) has long earned its reputation as anti-aging supplement in Chinese medicine and is one of the major components in recipes of many traditional Chinese anti-aging medicinal formulae (Lu et al., 1999; Wang et al., 2002; Deng et al., 2003). Experimentally, the polysaccharides extracts of Lycium barbarum has been shown to significantly prolong the half-death time for male Drosophila melanogaster (Xu et al., 2001). In addition, it can markedly attenuate hydroxyl radicals-induced lipid peroxidation in the liver of aged mice. Furthermore, it has been shown that polysaccharides extracts from Lycium barbarum can significantly reduce CCl₄-triggered liver toxicity (Kim et al., 1999). Also, it can exert protective effects against hyperthermia-induced damage in cultured seminoferous epithelium (Wang et al., 2002). Based on all of this evidence taken together, we have now recognized that polysaccharides extracts from Lycium barbarum exert protective effects to neurons against environmental toxins like β-amyloid (Aβ) peptides in AD.

The present inventors have achieved this invention by proving that the said compositions from aqueous extracts of the fruit of Lycium barbarum provide neuroprotective effects to Aβ peptide toxicity. Also, the technological platform of using phosphorylation of JNK and PKR will be an effective method for screening neuroprotective drugs from all kinds of natural or synthetic chemicals or formulations against β-amyloid peptides neurotoxicity.

### SUMMARY OF INVENTION

In accordance with the present invention, the foregoing objects and advantages have been readily obtained.

According to the invention, we have discovered that novel compositions which can be extracted and purified from Lycium barbarum is an effective agent for inhibition of Aβ-peptide induced neuronal cell death. In further accordance with the present invention, we have discovered that the purified compositions of the invention are effective agents for protecting neuronal cell death induced by Aβ-peptide. In still further accordance with the present invention, we have discovered methods for obtaining compositions having neuroprotection from Lycium barbarum; in addition, there are also provided formulations containing said composition. In further accordance with the present invention, we have discovered methods of purification of the invention composition from Lycium barbarum, and the characterization thereof.

### DETAILED DISCLOSURE OF THE INVENTION

The present invention provides novel therapeutic agents from the polysaccharides extracts from Lycium barbarum to prevent neuronal death and neuronal apoptosis in Aβ-peptide neurotoxicity and Alzheimer's disease. Also, the present invention provides a technological platform by using Western-blot analysis of PKR and JNK for drug screening to develop neuroprotective agent against neuronal loss in Alzheimer's disease.

We have demonstrated that the instant polysaccharides extracts from Lycium barbarum attenuate Aβ-peptide-triggered release of lactate dehydrogenase (LDH), caspases-3 and -2 activation, and PKR and JNK activation, by examining the morphological changes of neurons. This invention confirms that polysaccharides extracts from Lycium barbarum will be a good therapeutic and neuroprotective agent against Aβ-peptide neurotoxicity and neuronal death in Alzheimer's disease.

Development of the present invention has involved one or more of the following steps:

the step of comparing the release of lactate dehydrogenase (LDH), which serves as an index for cytotoxicity, from neurons exposed to Aβ-peptide and pre-treated with the aqueous or basic extracts (LBA and LBB, respectively) with an untreated control;

the step of comparing the activation of caspase-3 activity, which serves as a marker for neuronal apoptosis in neurons exposed to Aβ-peptide and pre-treated with the aqueous or basic extracts (LBA and LBB) with the control;

the step of comparing the activation of caspase-2 activity, which serves as a marker for Aβ-peptide neurotoxicity, in neurons exposed to Aβ-peptide and pre-treated with aqueous or basic extracts (LBA and LBB) with the control;

the step of comparing therapeutic window for the neuroprotective effects of LBA and lithium chloride in Aβ-peptide neurotoxicity;

the step of observing and comparing the morphological changes of neurons exposed to Aβ-peptide and pre-treated with LBA or heparin;

the step of examining the activation of JNK pathway in neurons exposed to Aβ-peptide and pre-treated with LBA and LBB;

the step of examining the activation of PKR pathway in neurons exposed to Aβ-peptide and pre-treated with LBA and LBB;

the step of isolation and purification of LBA and LBB into different fractions;

the step of comparing neuroprotective effects of different fractionated products from LBA by examining the Aβ-peptide-triggered caspase-3 activity;

the step of observing the morphological changes of neurons exposed to Aβ-peptide and pre-treated with the fractionated products from LBA;

the step of examining the activation of PKR pathway in neurons exposed to Aβ-peptide and fractionated product from LBA; and

the step of examining the neuroprotective effects of fractionated products from LBB on neurons exposed to Aβ-peptide.

### BRIEF DESCRIPTION OF THE DRAWINGS

FIG. 1 presents the elution profile of neuroprotective preparation from the aqueous extract of Lycium barbarum.

FIG. 2 presents the elution profile of neuroprotective preparation from the basic aqueous extract of Lycium barbarum.

FIGS. 3A to 3D collectively present the GC profile of neuroprotective preparation from the aqueous extracts of Lycium barbarum. Thus, FIG. 3A presents the GC profile of an aqueous extract (LBA; see Example 1); FIG. 3B presents the GC profile of LBA-A0, FIG. 3C presents the GC profile of LBA-A1 and FIG. 3D presents the GC profile of LBA-A2 (see Example 2).

FIGS. 4A to 4D collectively present the GC analysis of neuroprotective preparation from the basic aqueous extracts of Lycium barbarum. Thus, FIG. 4A presents the GC profile of an basic aqueous extract (LBB; see Example 1); FIG. 4B presents the GC profile of LBB-B0, FIG. 4C presents the GC profile of LBB-B1 and FIG. 4D presents the GC profile of LBB-B2 (see Example 2).

FIG. 5 shows the cytotoxicity analysis by the LDH activity assay.

FIG. 6 shows activity of caspase-3.

FIG. 7 shows the activity of caspase-2.

FIG. 8 shows the cytotoxicity analysis by the LDH activity assay in neuronal cell cultured after treatment with Aβ-peptide or LBB (0.1-500 μg/ml).

FIG. 9 shows caspase-3 activity after treatment with Aβ-peptide or LBB.

FIG. 10 shows the activity of caspase-2.

FIG. 11 shows the comparison of neuroprotective effects between LBA and LiCl.

FIG. 12 shows the comparison of neuroprotective effects of LBA, LBB and heparin on neuronal morphology.

FIG. 13 shows the Western-blot analysis of JNK and c-Jun in neurons treated with LBA and LBB.

FIG. 14 shows the comparison of neuroprotective effects between the specific JNK inhibitor SP 600125 and LBA.

FIG. 15 shows the Western-blot analysis of PKR and eIF2α in the neuroprotective effects of LBA and LBB.

FIG. 16 shows the effects of different fractionated products from LBA on Aβ-peptide neurotoxicity by examining caspase-3 activity.

FIG. 17 shows the neuroprotective effects of LBA-A2 against Aβ-peptide neurotoxicity in term of neuronal morphology.

FIG. 18 shows the Western-blot analysis of LBA-A2.

FIG. 19 shows the neuroprotective effects of fractionated products (LBB-B1) from LBB on Aβ-peptide neurotoxicity by examining the caspase-3 activity.

FIG. 20 shows the neuroprotective effects of fractionated products (LBB-B2) from LBB on Aβ-peptide neurotoxicity by examining the caspase-3 activity.

### DETAILED DESCRIPTION OF THE INVENTION

In accordance with the present invention, there are provided compositions in substantially purified form, said compositions characterized as:
(1) water soluble polyanionic polysaccharide-containing extracts comprising arabinose, galactose, glucose, xylose, rhamnose, mannose, glucuronic and glactouronic acid as analyzed by gas chromatography;
(2) able to inhibit Aβ-amyloid induced neuronal cell death;
(3) having substantially little or no in vivo toxicity when topically applied to a mammal at a concentration of about 0.1 to 500 μg/ml; and
(4) containing substantially no ethanol soluble components.
   The invention compositions can be further characterized as:
(5) having a molecular mass less than 500 kDa;
(6) being stable to exposure to temperature in the range of about 95-100° C. for 4 hours;
(7) being substantially insoluble in methanol, ethanol, butanol, acetone and chloroform;
(8) having an elemental content of about 35.5 to 39.6% carbon, about 5.4 to 5.5% hydrogen, about 2.7 to 5.5 % nitrogen and about 0.9 to 3.0% sulfur;
(9) containing 15 to 84% (w/w) carbohydrate, expressed as glucuronic and galacturonic acids;
(10) containing 8 to 93% (w/w) uronic acid, expressed as galacturonic acid; and
(11) containing 0 to 8% (w/w) uronic acid, expressed as glucuronic acid;
(12) being effective for the inhibition of Aβ-amyloid induced neuronal cell death

The novel compositions of the present invention can be prepared by a variety of methods for both extraction and purification. Such methods include the one described in the Example of the present specification. Briefly, the dry fruit is allowed to soak in ethanol or another lower alcohol which is then driven off by heating taking substantially all of the alcohol soluble components with it. The residue crushed into a powder and extracted with water resulting in an aqueous solution (which can be freeze-dried if desired) and a second residue. One extract of the invention (LBA) can be realized by adding ethanol to the aqueous solution to form a precipitate, which is optionally further extracted with butanol and/or methanol. A second extract of the invention (LBB) can be realized by combining the second residue with an aqueous base, such as, for instance, sodium hydroxide, resulting in a basic aqueous solution which can be dialysized and concentrated, followed by adding ethanol to the aqueous solution to form a precipitate, which is optionally further extracted with methanol. Both extracts can be fractionated by column chromatography using gradient aqueous base as an eluent.

In another approach, for example, the composition can be obtained from cells of the dry Lycium barbarum fruit by purifying the invention composition by contacting an extract from Lycium barbarum with an anion exchange material which selectively binds negatively charged materials, and recovering the invention composition from the anion exchange material. The present invention is also directed to pharmaceutical formulations suitable for the inhibition of Aβ-peptide induced neuronal cell death thereof, which contains an effective amount of the invention composition, with or without an appropriate pharmaceutically acceptable carrier therefor. Any compatible carrier can be employed which is suitable for the type of dosage form employed. Such formulations include solid dosage forms such as tablets, capsules, cachets, pellets, pills, powders or granules, topical dosage forms such as solutions, powders, fluid emulsion, fluid suspensions, semi-solids, ointments, pastes, reams, gels or jellies and foam, and parenteral dosage forms which include solutions, suspensions, emulsions or dry powers. The means and methods for administration are known in the art and those skilled can refer to various pharmacological references for guidance. Some examples are “Modem Pharmaceutics”, Banker & Rhodes, Marcel Dekker, Inc. 1979 and “Goodman & Gilman's The Pharmaceutical Basis of Therapeutics”, MacMillan Publishing Co. Appropriate dosage amounts can be readily determined by those of skill in this art.

The composition of the present invention is preferably present in a purified form when administered. When invention compositions are obtained by extraction from Lycium barbarum, it is desirable to separate soluble extract from (residual) particulate matter by appropriate means (e.g., filtration, centrifugation, or other suitable separation techniques). Separation of solid materials may be performed one or more times during the extracting process. The utility of invention compositions as a therapeutic agent is enhanced by greater purification. Greater doses may be necessary when less pure forms of the extract are employed.

The invention compositions are preferably substantially free from heavy metals, contaminating plant materials, contaminating microorganisms, oxalic acid or precursors of oxalic acid or any other contaminants which may be present in a preparation which can be derived from plant material.

Although isolation of the invention compositions from the fruit of Lycium barbarum is the presently most practical method for obtaining such materials, the present invention also contemplates obtaining such materials from other sources such as other plants which may contain recoverable amount of compositions having the properties described herein. Other plants contemplated include species within the family of Solanaceae, of which Lycium is a member. It is also possible that invention compositions could be obtained by culturing plant cells, such as Lycium barbarum cells, in vitro and extracting the active ingredients from the cells or recovering the active ingredients from the cell culture medium.

As used herein, the term “extract” means the active ingredients isolated from the fruit or other parts of Lycium barbarum or other natural sources including but not limited to all varieties, species, hybrids or genera of the plant regardless of the exact structure of the active ingredients, from or method of preparation or method of isolation. The term “extract” is also intended to encompass salts, complexes and/or derivatives of the extract which possess the above-described biological characteristics or therapeutic indication. The term “extract” is also intended to cover synthetically or biologically produced analogs and homologs with the same or similar characteristics yielding the same or similar biological effects of the present invention.

The purified composition contemplated for use herein include purified extract fractions having the properties described herein from any plant or species, preferably Lycium barbarum, in natural or in variant form, and from any source, whether natural, synthetic, or recombinant. Also, included within the scope of the present invention are analogs and homologs of the above-described purified compositions.

The present invention also contemplates the use of synthetic preparations having the characteristics of invention compositions. Such synthetic preparations could be prepared based on the chemical structure and/or functional properties of the above-described compositions of the present invention. Also contemplated are analogs and homologs of the chemical structure of the invention compositions and having the functional properties of compositions according to the present invention.

As used herein, reference to “analogs and homologs” of the invention compositions embraces compounds which differ from the structure of invention compositions by as little as the addition and/or replacement and/or deletion of one or more residues thereof. Such compounds in all instances, however, have substantially the same activity as invention compositions. Thus, “analogs” refers to compounds having the same basic structure as invention compositions, but differ in one or more residues; “homologs” refers to compounds which differ from invention compositions by the addition and/or deletion and/or replacement of a limited number of residues. Based on the in vitro data presented herein, a concentration of about 0.1 to 500 μ/ml of invention composition (see Example 7-12) is expected to be effective for topical application in preventing the Aβ-induced neuronal cell(s) death. Suitable amounts for other modes of administration can be readily determined by those skilled in this art using standard, routine techniques.

### EXAMPLES

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

### Example 1

### Extraction of Invention Composition from Lycium barbarum

The dried fruit of Lycium barbarum (10 kg) were soaked in 95% ethanol (10 L) for 120 h. The resultant residue was filtered, dried and ground into small pieces with a blender. Distilled water (50 L) was added and the suspension was simmered at 95-100° C. for 3 h. The extract was decanted to a clean container and the Lycium barbarum was extracted two more times with distilled water under the same conditions. The extracts were poured through a cotton cloth to remove insoluble materials (residue). The volume of the clarified extracts was reduced to about 20 litres by a rotary evaporator. The condensed extract was freeze-dried. A total of 1800 g of dark brown dried powder was obtained.

The neuroprotective component in the resulting aqueous extract was precipitated by ethanol. To achieve this, 1800 g of the freeze-dried aqueous extract was dissolved in 20 L of water, and ethanol was added to a final concentration of 90% (vol/vol). After leaving the mixture at 4° C. for 24 h, the precipitate was obtained by filtration through cotton and washed with 2×1.5 L of butanol, followed by 2×1.5 L of methanol. This yielded 1000 g of dark brown powder, hereinafter designated LBA, after being freeze-dried

The residue was further incubated with a 5% sodium hydroxide solution (15 L) for 24 h. The resulting basic extracts were poured through a cotton cloth to remove insoluble materials. The volume of the basic extracts was exhaustively dialyzed (MW cutoff 3000-5000 Da) with running water for 60 h. The volume of the dialyzate was reduced to about 8 L by a rotary evaporator. The neuroprotective component in the resulting basic aqueous extract was precipitated by ethanol. Thus, ethanol was added to the dialyzate to a final concentration of 90% (vol/vol). The mixture was left for 24 h and the precipitate was obtained by filtration through cotton and washed with 2×1.5 L of methanol. This yielded 75 g of dark brown powder, hereinafter designated LBB.

### Example 2

### Purification of Extract Fraction by Column Chromatography

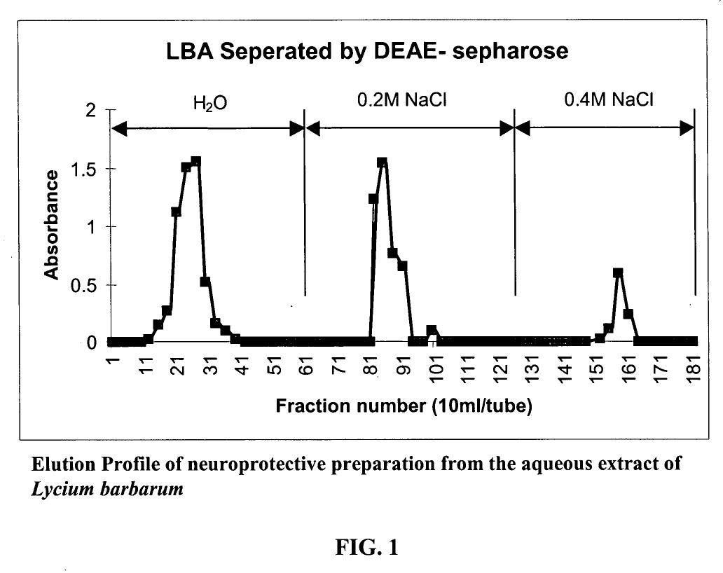

The neuroprotective component of the aqueous extract was further purified by gel filtration column chromatography. An aqueous solution of LBA (2 g in 3 ml) was applied to a DEAE Sepharose Fast Flow column (100×2.6 cm) and eluted first with distilled water followed by 0.2M and 0.4M aqueous sodium hydroxide solution (gradient elution). Fractions of 10 ml were collected and the amount of carbohydrate in the collected fractions was monitor by phenol-sulfuric acid assay using water as the standard (see FIG. 1). These separated fractions were concentrated by a rotary hevaporator (about 10 ml) and exhaustively dialyzed (MW cutoff 3000-5000 Da) with running water for 60 h followed by being freeze-dried to give three fractions, hereafter designated LBA-A0, LBA-A1 and LBA-A2.

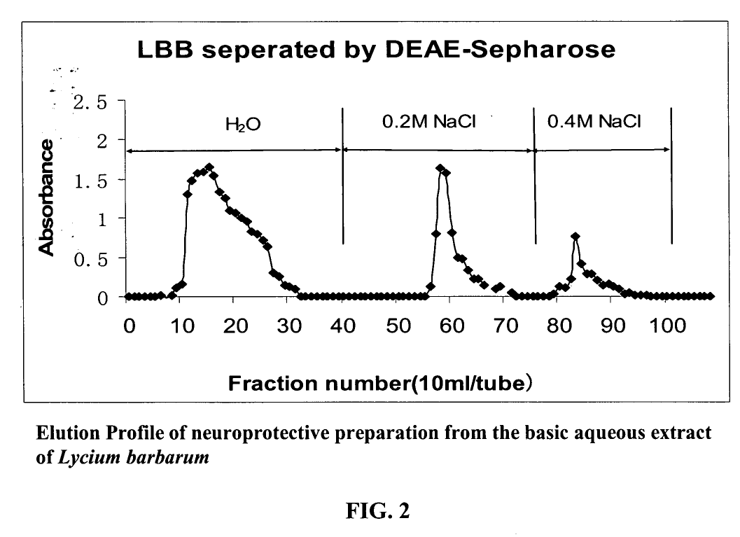

The neuroprotective component of the basic aqueous extract was further purified by a similar method. Thus, an aqueous solution of LBB (2 g in 5 ml) was applied to a DEAE Sepharose Fast Flow column (100×2.6 cm) and eluted first with distilled water followed by 0.2M and 0.4M aqueous sodium hydroxide solution (gradient elution) to give three fractions, hereinafter designated LBB-B0, LBB-B1 and LBB-B2 (see FIG. 2).

### Example 3

### Chemical Nature of Neuroprotective Compound from Lycium barbarum

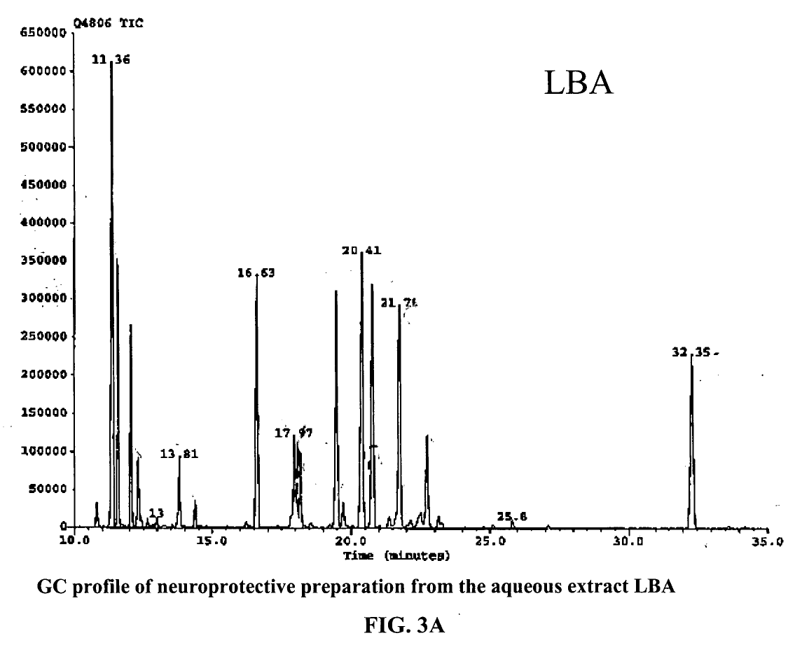

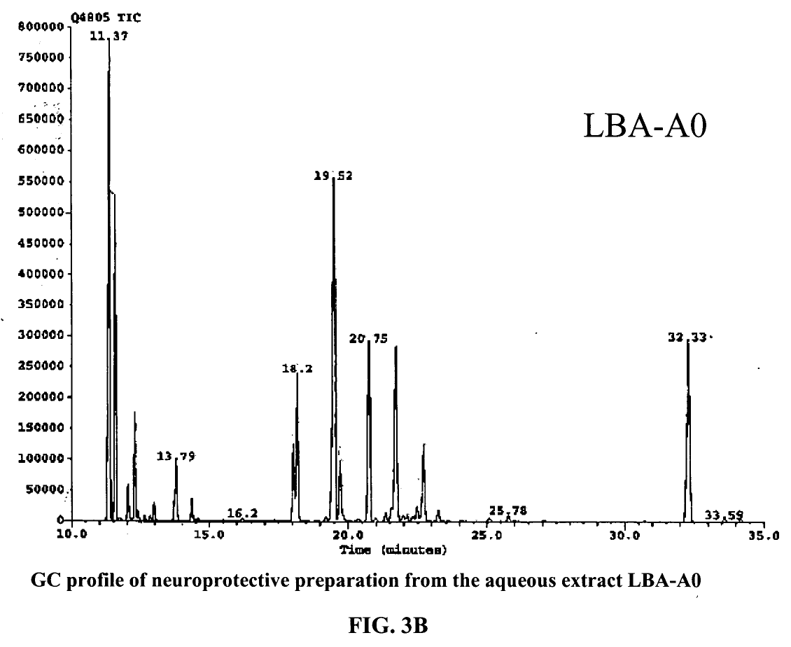

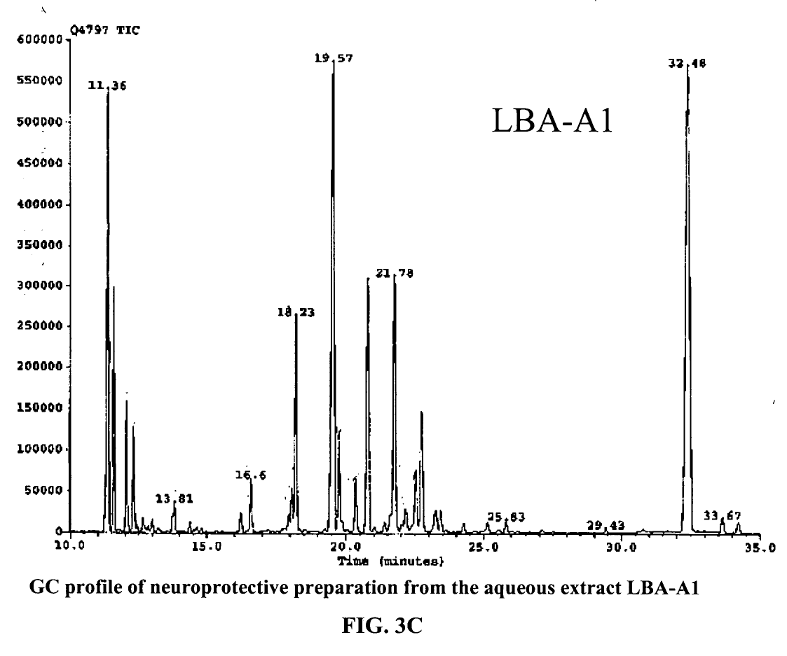

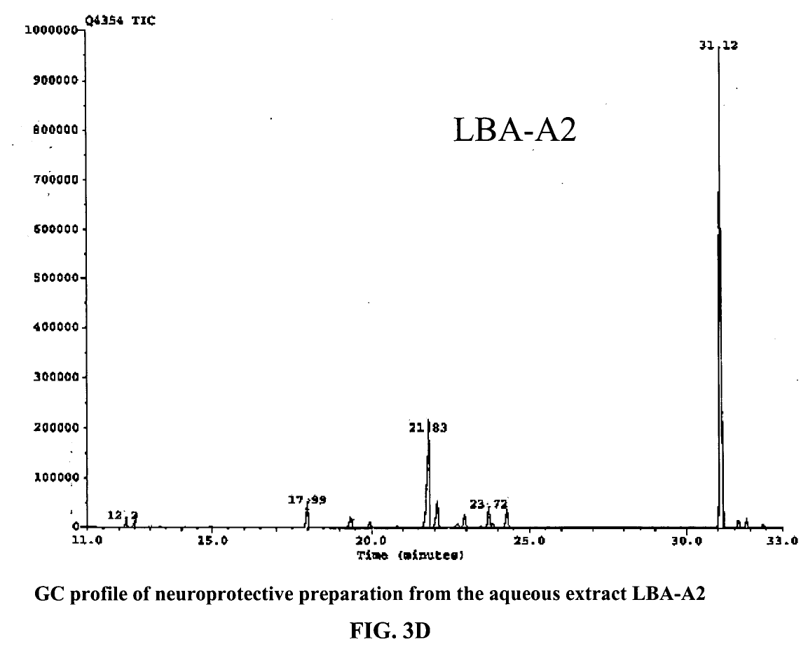

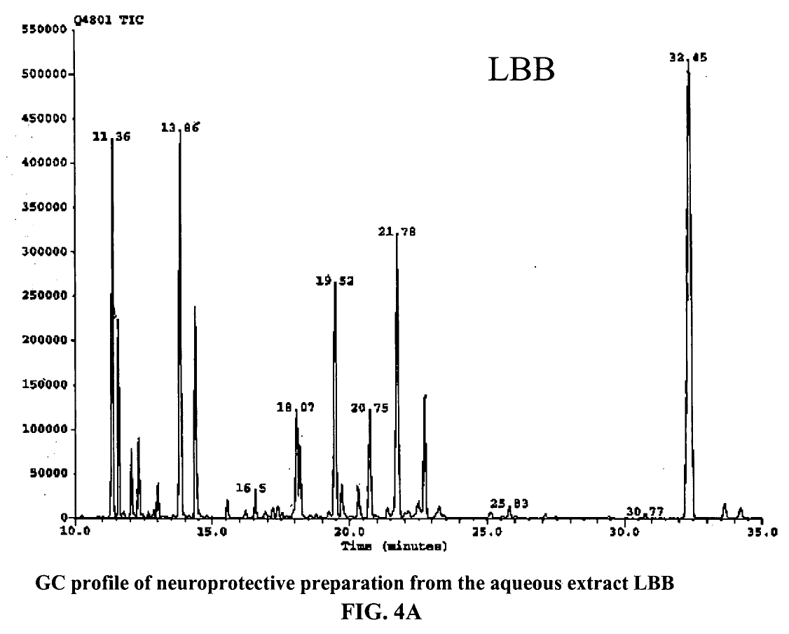

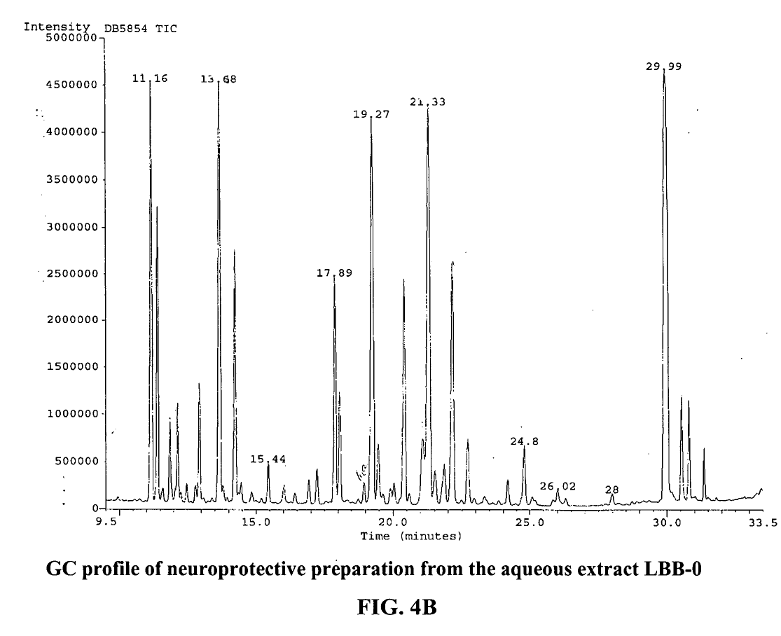

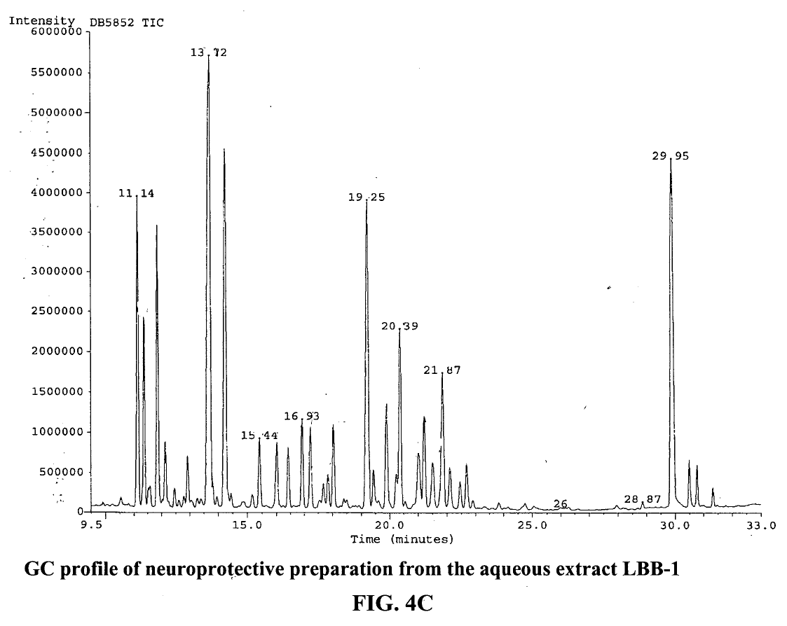

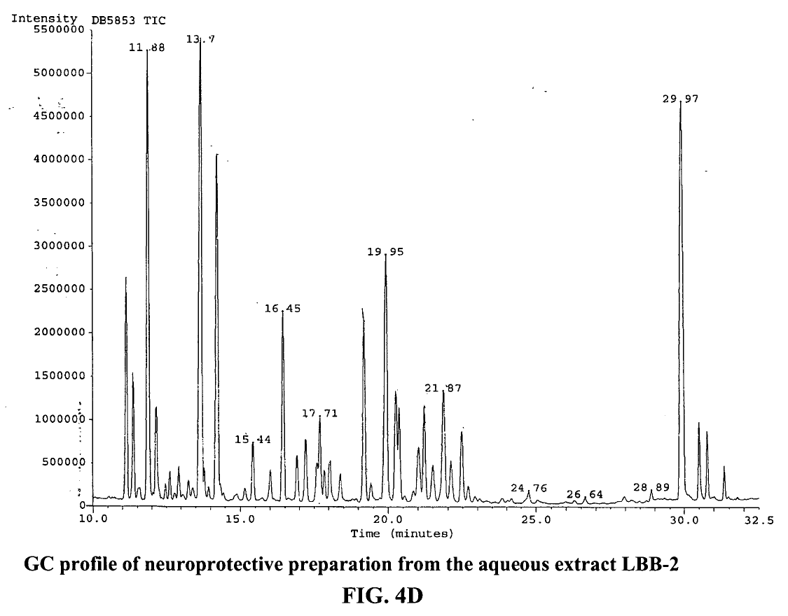

The chemical nature of the aqueous extract and basic aqueous extract neuroprotective fractions was investigated by different chemical tests. The glycosyl compositions analysis was performed by combined gas chromatography/mass spectrometry (GC/MS) of the per-O-trimethylsilyl (TMS) derivatives of the monosaccharide methyl glycosides produced from the sample by acidic methanolysis. To achieve this, methyl glycosides were first prepared from a portion of each dry sample by methanolysis in 1 M HCl in methanol at 80° C. (18-22 h), followed by re-N-acetylation with pyridine and acetic anhydride in methanol. The samples were then per-O-trimethylsilylated by treatment with Tri-Sil at 80° C. (30 min). The results confirmed that the neuroprotective aqueous extract fraction from Lycium barbarum is different from the basic aqueous extract fraction (see FIGS. 3 and 4).

Elemental, infrared, NMR and other spectroscopic analytical means can also be employed to characterize the active compound.

### Example 4

### Reduction of β-amyloid Peptide-induced Release of Lactate Dehydrogenase (LDH) from Neurons by the Aqueous Extract LBA

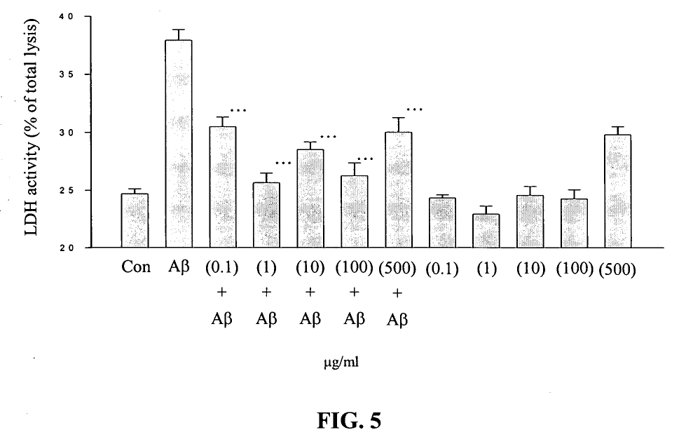

To examine the effects of the aqueous extract LBA from Lycium barbarum on Aβ-peptide-induced neurotoxicity, neurons were pretreated with LBA (ranging from 0.1 μg/ml to 500 μg/ml) for 1 hour, followed by a 24-hour exposure to Aβ₂₅₋₃₅ peptide (25 μM). For cytotoxicity analysis, the extracellular concentration of LDH was measured. FIG. 5 shows the percentage of total LDH release in the various treatment groups expressed as mean±SE from at least three independent experiments. For the control group, the release of LDH representing natural neuronal death was 24.7±0.45% of the total LDH release. Neurons exposed to Aβ-peptide increased the release of LDH to 38.0±0.92%. Different concentrations of LBA were able to significantly (***p<0.001 by one-way ANOVA, Student-Newman-Keuls method) reduce the LDH release after the Aβ-peptide incubation. The results demonstrated that LBA exhibited cytoprotective effect against Aβ-peptide toxicity.

### Example 5

### Reduction of β-amyloid Peptide-triggered Activation of Caspase-3, a General Apoptosis Marker, in Neurons by the Aqeous Extracts LBA

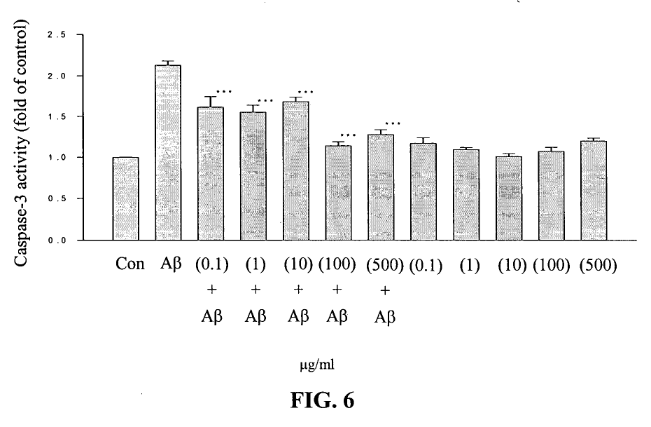

Caspase-3 has long been regarded as an index for neuronal apoptosis. Therefore, the colorimetric caspase-3 activity assay was performed at the concentrations stated in FIG. 5, and neuronal apoptosis was determined by the colorimetric caspase-3 activity assay by measuring the absorbance (at 405 nm) of the yellow product (pNA) cleaved from the substrate. Results (***p<0.001 vs. the group treated with Aβ-peptide alone, by one-way ANOVA, Tukey test) are expressed as mean±SE from at least three independent experiments. As shown in FIG. 6, neurons exposed to Aβ-peptide displaced a two-fold increase in caspase-3 activity. However, neurons exposed to different concentrations of LBA (0.1-500 μg/ml) significantly attenuated activation of caspase-3 triggered by Aβ-peptide. LBA at 100 μg/ml showed the best neuroprotective effect since it could reduce caspase-3 activity from 2.13 fold to 1.14 fold when compared with the Aβ-peptide treated group. Neurons exposed to the aqueous extract LBA per se did not induce a significant increase in caspase-3 activity.

### Example 6

### Reduction of β-amyloid Peptide-triggered Activation of Caspase-2 in Neurons by the Aqeous Extracts LBA

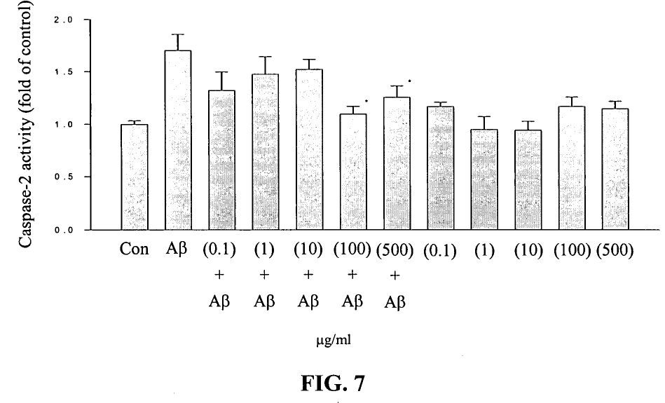

Caspase-2 is an important index for neuronal apoptosis for β-amyloid peptide neurotoxicity. It has been reported that Aβ-peptide triggered activation of caspase-2. Also, it has been suggested that the roles of caspase-2 in Aβ-peptide neurotoxicity is more significant than that of caspase-3. Therefore, we used the colorimetric method to determine caspase-2 activity. For caspase-2 activity, the trend was similar to that of caspase-3. Neurons exposed to Aβ-peptide triggered a 1.6-fold increase in caspase-2 activity. LBA at 100 and 500 μg/ml significantly inhibited Aβ-peptide-triggered activation of caspase-2 from 1.70 fold to 1.10 fold and 1.26 fold, respectively (see FIG. 7; *p<0.05 vs. the group treated with Aβ-peptide alone, by one-way ANOVA, Student-Newman-Keuls method).

### Example 7

### Reduction of β-amyloid Peptide-induced Release of Lactate Dehydrogenase (LDH) from Neurons by the Basic Extract LBB

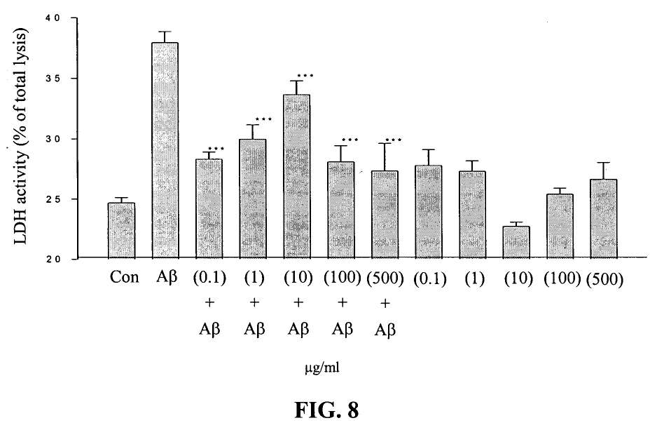

The effects of the basic extract LBB from Lycium barbarum on Aβ-peptide-induced neurotoxicity were also investigated using the same approaches as for LBA. For cytotoxicity analysis, the extracellular concentration of LDH was measured. FIG. 8 shows the percentage of total LDH release of various treatment groups. Neurons pre-treated with LBB of different concentrations (0.1-500 μg/ml) significantly reduced the LDH release triggered by Aβ-peptide. The results are expressed as mean±SE from at least three independent experiments (***p<0.001 vs. the group treated with Aβ-peptide alone, by one-way ANOVA, Student-Newman-Keuls method) showed that LBB exerted protective effects against Aβ-peptide-induced cytotoxicity.

### Example 8

### Reduction of β-amyloid Peptide-triggered Activation of Caspase-3 in Neurons by the Basic Extracts LBB

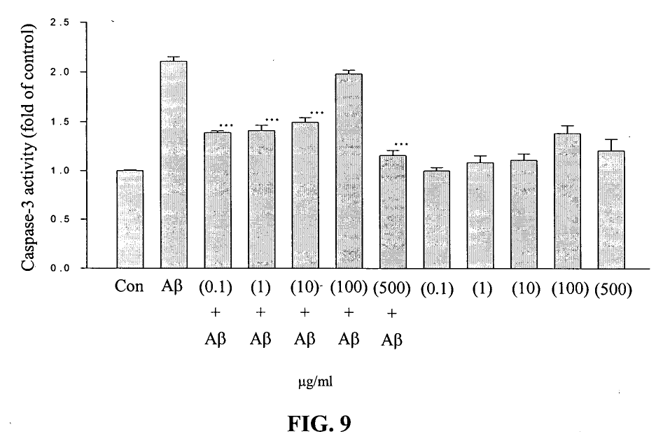

Caspase-3 is a general apoptosis marker. For apoptosis analysis, the colorimetric caspase-3 activity assay (at 405 nm) was performed. LBB at low concentrations (0.1, 1 and 10 μg/ml) and at 500 μg/ml significantly decreased Aβ-peptide-stimulated caspase-3 activity to only 1.3-1.5-fold of control (see FIG. 9; *p<0.001 vs. the group treated with Aβ-peptide alone, by one-way ANOVA, Tukey test). However, it did not alter Aβ-peptide toxicity at 100 μg/ml. The results suggested that LBB elicits biphasic neuroprotective effects to attenuate activation of caspase-3.

### Example 9

### Reduction of β-amyloid Peptide-triggered Activation of Caspase-2 in Neurons by the Aqueous Extracts LBA

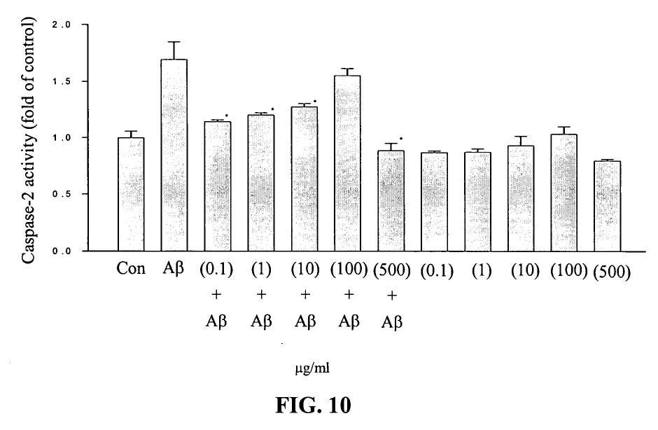

Caspase-2 is an important index for neuronal apoptosis for β-amyloid peptide neurotoxicity. For caspase-2 activity, the trend was similar to that of caspase-3. We also used colorimetric method to determine caspase-2 activity. Apart from 100 μg/ml, LBB was able to significantly attenuated Aβ-peptide-induced caspase-2 activity to about 1.0 to 1.3-fold of control (FIG. 10, *p<0.05 vs. the group treated with Aβ-peptide alone, by one-way ANOVA, Student-Newman-Keuls method). LBB from 1 to 500 μg/ml per se did not trigger a significant increase in caspase-2 activity.

### Example 10

### Comparison for the Therapeutic Window of Neuroprotective Protective Effects Between LBA and Lithium Chloride (LiCl)

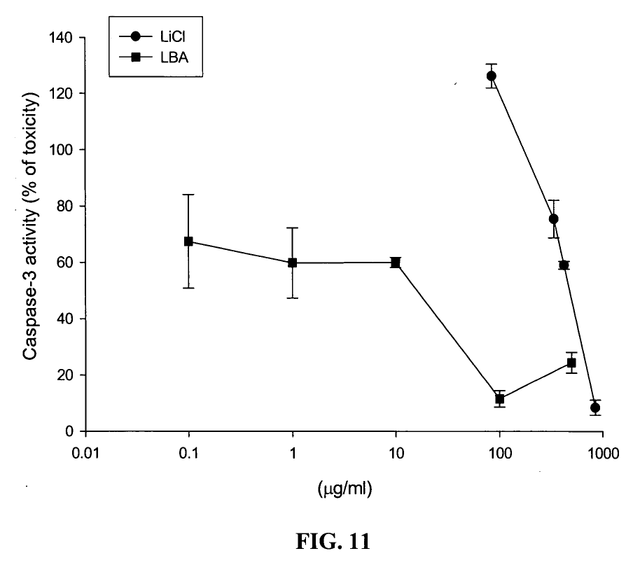

LiCl has been shown to protect neurons against Aβ-peptide toxicity. It is also a well-known Western medicine for the treatment of manic-depressive bi-polar disorder. We have demonstrated the neuroprotective effects of LBA and LBB against Aβ-peptide-induced apoptosis and the purpose of this Example was to find out how effective is LBA in neuroprotection by comparing it with the well-known neuroprotective drug, LiCl. Neurons were pretreated with either LiCl (2 mM to 20 mM) or LBA (0.1 μg/ml to 500μ/ml) for 1 hour, and then exposed to Aβ₂₅₋₃₅ peptide (25 μM) for 24 hours. Caspase-3 activity assay was used to determine the protective effects against Aβ-peptide toxicity. FIG. 11 shows the caspase-3 activity (in terms of percent toxicity, calculated as (s.a._{of Aβ-/LBA-/SP-treated}−s.a._{control})÷(s.a._{Aβ-treated}−s.a._{control}); expressed as mean+/−SE from at least 3 independent experiments) after LBA and LiCi treatment. The unit of concentration of LiCl was converted from mM to μ/ml for the purpose of comparison with LBA. LiCi was toxic to neurons at about 85 μg/ml (2 mM). The toxicity decreased when the concentration of LiCi increased and it reached the lowest value (8.5%) when LiCi was used at 850 μ/ml (20 mM). Accordingly, LiCl could effectively reduce Aβ-peptide-stimulated caspase-3 activity in the range of 340 μg/ml to 850 μg/ml (i.e. 8 mM to 20 mM). LBA exerted protective effects for neurons against Aβ-peptide toxicity from very low concentration to high concentration (0.1 μg/ml to 500 μg/ml). The toxicity was significantly lowered to 11.6% when LBA was used at 100 μg/ml. The results in FIG. 11 indicate that LBA has a wider therapeutic window than that of LiCl.

### Example 11

### Comparison for the Neuroprotective Effects of LBA, LBB and Heparin by Morphological Changes of Neurons

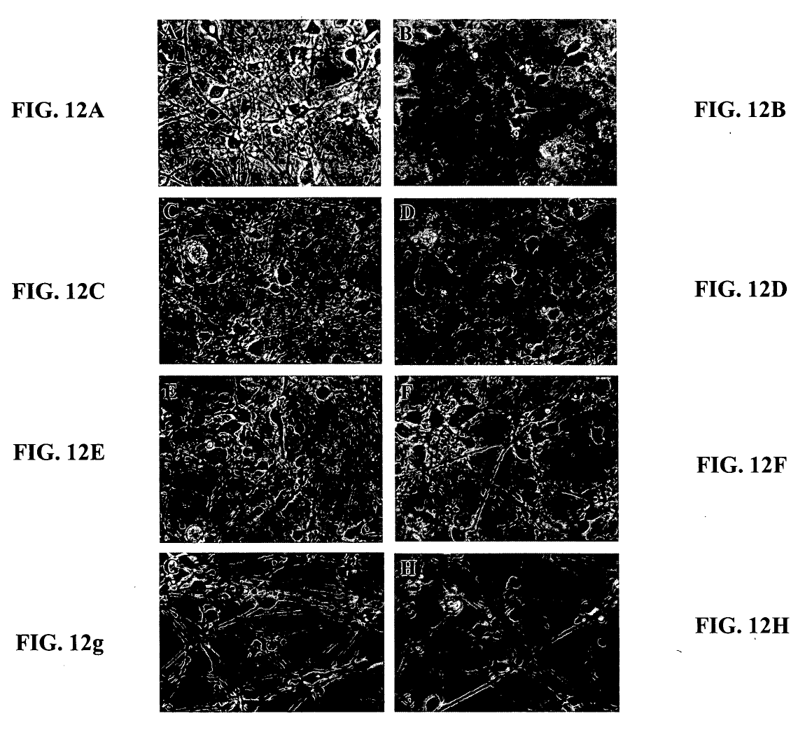

Heparin is a well-defined glycoprotein that has been demonstrated to attenuate Aβ-peptide neurotoxicity. Since LBA and LBB contain both carbohydrate and amino acid, we use heparin to make a comparison of their protective effects. Neurons were pre-incubated with either heparin (1 μM), LBA (100 μg/ml) or LBB (500 μg/ml) for 1 hour, followed by a 24-hour exposure to Aβ peptide (25 μM). The morphology of neurons after different treatments is shown in FIG. 12, in which (A) Control, (B) Aβ-peptide alone, (C) LBA (100 μg/ml), (D) LBA (100 μg/ml)+Aβ, (E) LBB (500 μg/ml), (F) LBB (500 μg/ml)+Aβ, (G) Heparin (1 μM) and (H) Heparin (1 μM)+Aβ. In the control (A), neurons were in good condition as revealed by the fine neurite network and the round cell bodies. In B, neurons treated with Aβ-peptide for 24 hours undergo apoptosis, as shown by the destruction of the fine neurite network. As shown in D and F, neurons were still in good condition, which indicated that pretreatment of neurons with either LBA or LBB inhibited the Aβ-peptide-induced apoptosis. G and H were neurons pre-incubated with heparin, and then without and with Aβ-peptide exposure. Both pictures show elongated neurons. It is obvious that neurons treated with heparin had a great morphological difference to those treated with LBA or LBB. Heparin has already been shown to have neuroprotective effects in Aβ-peptide toxicity by other laboratories. We also found that LBA and LBB possess neuroprotective effects against Aβ-peptide toxicity. Based on the morphology of neurons, the mode of neuroprotection of LBA and LBB is somewhat different to that of heparin.

### Example 12

### Reduction of β-amyloid Peptide Triggered Activation of Stress Kinase, c-Jun N-terminal Kinase (JNK), by Aqueous Extract LBA

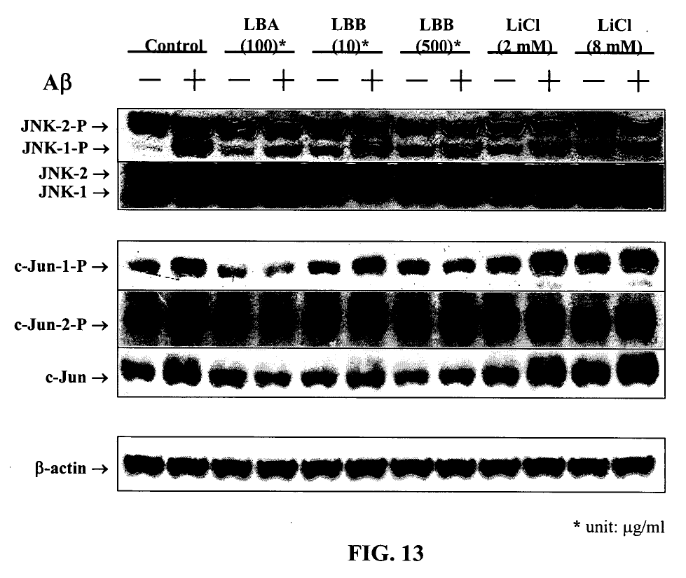

JNK is one of the stress kinases involved in the processes of neuronal apoptosis in many types of stress responses. It has also been reported that JNK is involved in Aβ-peptide neurotoxicity. Therefore, we examined how the aqueous extract from Lycium barbarum LBA modulates the JNK kinase pathway by Western-blot analysis. Upon Aβ-peptide stimulation, JNK is activated by phosphorylation. Activated JNK further phosphorylates its substrate, c-Jun, which will in turn target on downstream effectors, causing apoptosis. Neurons were pretreated with LBA (100 μg/ml), LBB (10 μ/ml and 500 μ/ml) or LiCi (2 mM and 8 mM) for 1 hour, followed by a 4-hour or a 6-hour exposure to Aβ₂₅₋₃₅ peptide (25 μM). Proteins extracted were subjected to Western-blot analysis (FIG. 13) to detect the level of phospho-JNK, phospho-c-Jun, total JNK, total c-Jun and β-actin. β-actin was used as the control. As shown in FIG. 13, the levels of phospho-JNK-1 were significantly up-regulated by Aβ-peptide. Similar effects were also observed in phospho-c-Jun-I and phospho-c-Jun-II which were increased when neurons were exposed to Aβ-peptide alone. Pretreatment with LBA at 100 μg/ml markedly decreased the levels of these three phosphorylated proteins (FIG. 13). However, pretreatment of LBB at either low or high concentration did not reduce the levels of phospho-JNK-1 and phospho-c-Jun-I. The protein levels of total JNK, total c-Jun and β-actin were unchanged even after LBA and LBB treatment.

### Example 13

### Comparison for the Therapeutic Window of Neuroprotective Effects Between the Specific JNK Inhibitor SP 600125 and LBA

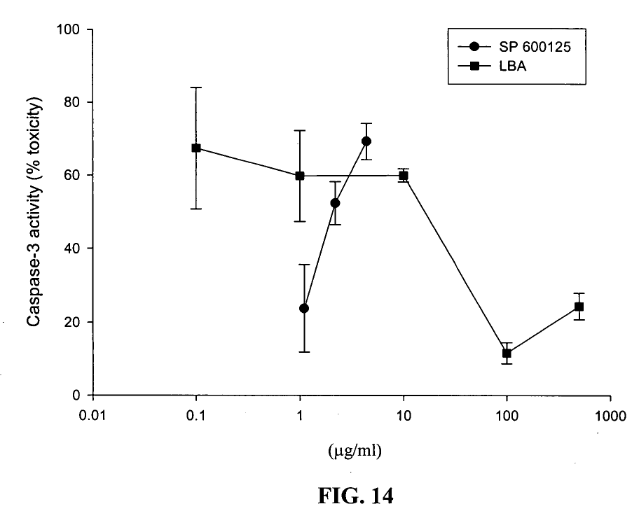

Western-blot analysis results indicate that LBA can attenuate activation of JNK pathway. It has been reported that specific inhibitor of JNK, SP600125, also exerts protection for neurons against Aβ-peptide toxicity. We therefore compared the therapeutic window of SP600125 with LBA to elucidate their effectiveness in neuroprotection. Neurons were pretreated with either SP600125 (5 μM to 20 μM) or LBA (0.1 μg/ml to 500 μg/ml) for 1 hour, and then exposed to Aβ-peptide (25 μM) for 16 hours. Caspase-3 activity assay was done to investigate their protective effects against Aβ-peptide toxicity. Thus, after treatment, colorimetric caspase-3 activity assay was done to examine the level of apoptosis. Caspase-3 activity was expressed as % toxicity. Results are expressed as mean±SE from at least three independent experiments. % toxicity is calculated as: (s.a._{of Aβ-/LBA-/SP-treated}−s.a._{control})÷(s.a._{Aβ-treated}−s.a._{control}). FIG. 14 shows the caspase-3 activity (in terms of percentage toxicity) after SP600125 and LBA treatment. The unit of concentration of SP600125 was converted from mM to μg/ml for the purpose of comparison with LBA. SP600125 reduced the toxicity to 23.8% at 1.1 μg/ml (5 μM). However, the toxicity reached 69.3% when SP600125 was used at 4.4 μg/ml (20 μM). The toxicity was 100% when using SP600125 at more than 20 μM (data not shown). SP600125 was effective from 1.1 μg/ml to 4.4 μ/ml; the range is very narrow when compared with that of LBA.

### Example 14

### Reduction of β-amyloid Peptide Triggered Activation of Stress Kinase, Double-stranded RNA-dependent Protein Kinase (PKR), by Aqueous Extract LBA

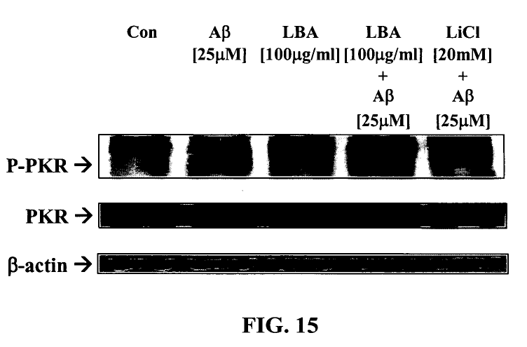

The reduction of β-amyloid peptide triggered activation of stress kinase, double-stranded RNA-dependent protein kinase (PKR), by aqueous extract LBA was investigated and the results are shown in FIG. 15.

### Example 15

### Neuroprotective Effects of Fractionated Product from Aqueous Extract LBA Against β-amyloid Peptide Neurotoxicity

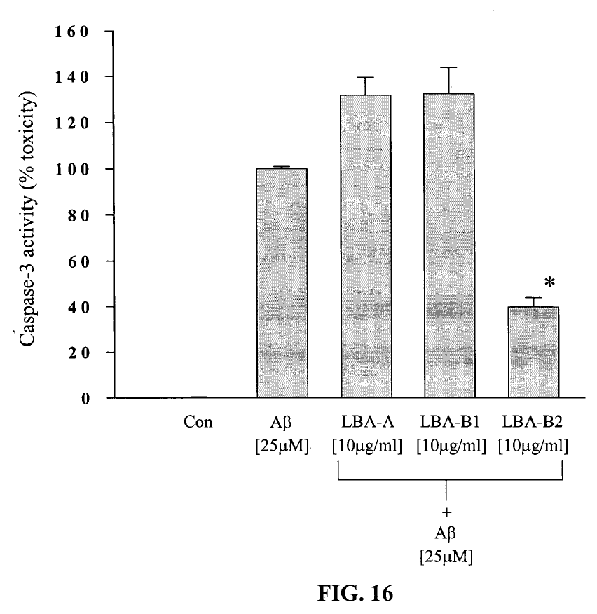

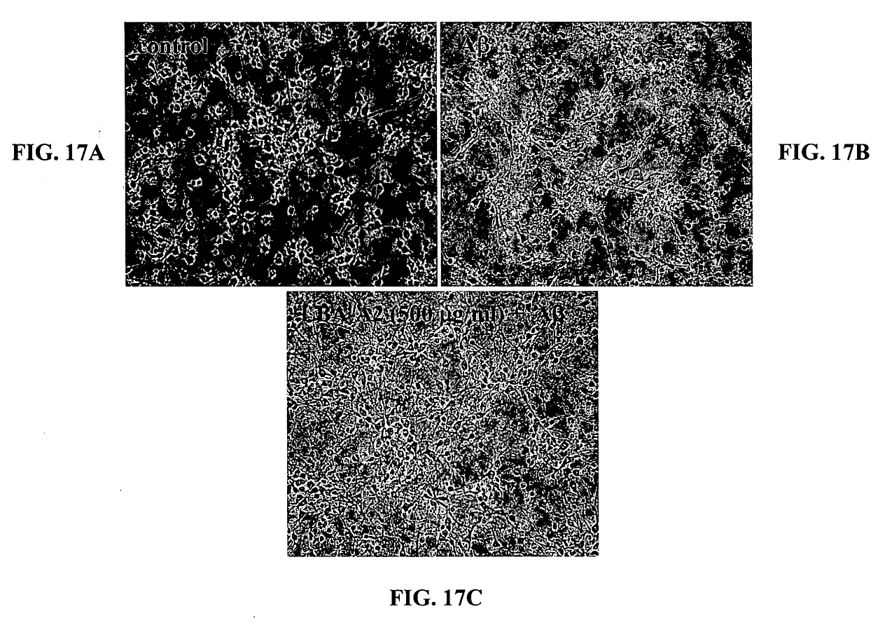

Since the aqueous extract LBA from Lycium barbarum exhibits neuroprotective effects against Aβ neurotoxicity, we conducted a further investigation of what is an active fraction responsible for the neuroprotection. We separated different fractions by ion-exchange chromatography. Activity of caspase-3 served as an index for neuronal apoptosis. Among three different fractions, only the LBA-A2 fraction attenuated Aβ-peptide-triggered caspase-3 activity (FIG. 16 where results are expressed as mean±SE from at least three independent experiments. *p<0.001 vs. the group treated with Aβ-peptide alone, by one-way ANOVA, Tukey test.). The results indicate that the 0.4 M eluted product from the extract of Lycium barbarum contains the neuroprotective component against Aβ peptide neurotoxicity. The biochemical analysis of caspase-3 activity was confirmed by morphological examination (FIG. 17). Upon exposure to Aβ peptide, neurites were broken and neurons were undergoing apoptosis. However, neurons pre-treated with LBA-A2 preserved fasciculation of neurites and integrity of neurons.

### Example 16

### Active Fraction from the LBA Extracts of Lycium barbarum Reduce the Activation of Stress Kinase PKR Pathway in Neurons Triggered by β-amyloid Peptide

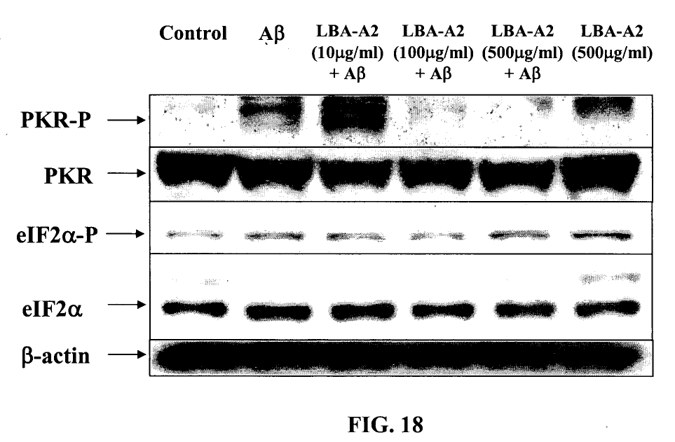

Since extracts from Lycium barbarum attenuated Aβ-triggered phosphorylation of PKR, we had further examined whether the neuroprotective effects of fractionated product LBA-A2 was mediated via PKR pathway by Western-blot analysis. As shown by our previous results, neurons exposed to Aβ peptide have a marked increase in PKR as well as eIF2α phosphorylation. While pre-treatment of 10 μg/ml of LBA-A2 enhanced the phosphorylation of PKR in neurons, LBA-A2 at 100 and 500 μg/ml markedly reduced phosphorylation of PKR 4 h after exposure to Aβ peptide (FIG. 18). Neurons were pre-treated with LBA-A2 at 10, 100 or 500 μg/ml for 1 h prior to the exposure to Aβ-peptide (25 μM) for 4 h. Protein was extracted for Western-blot analysis of phosphorylated PKR and eIF2α. The protein levels of total normal PKR, eIF2α and β-actin remained unchanged after the treatment.

### Example 17

### Neuroprotective Effects of Fractionated Product from Aqueous Extract LBB Against β-amyloid Peptide Neurotoxicity

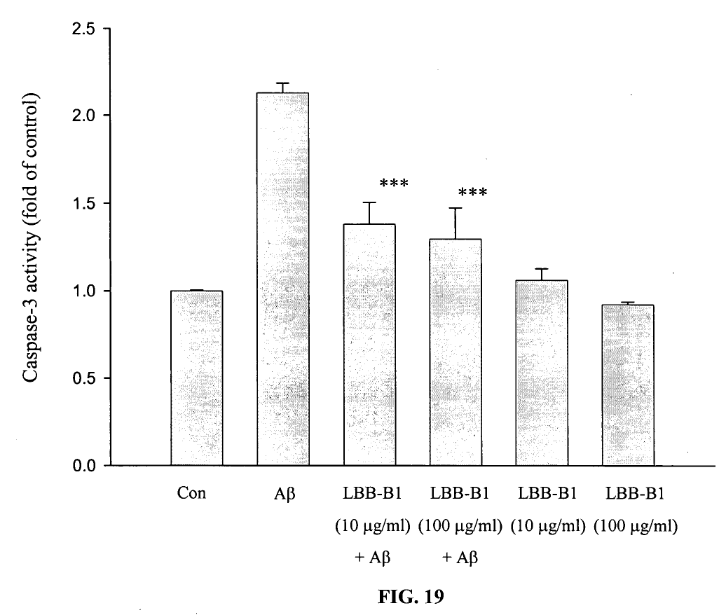

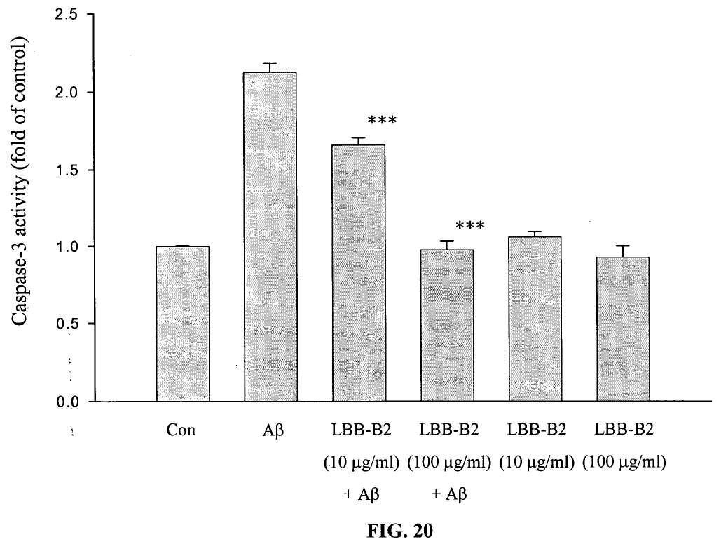

For apoptosis analysis, the activity of caspase-3 serves as an index for neuronal apoptosis. Primary cultured neurons pre-treated with LBB-B1 at either 10 or 100 μg/ml significantly attenuated Aβ-peptide neurotoxicity (FIG. 19). LBB-B1 per se has no neurotoxicity. Similar to LBB-B1, pretreatment of LBB-B2 also exhibit significant neuroprotective effects against Aβ neurotoxicity (FIG. 20). A hundred microgram per micro-liter exerted a nearly 100% neuroprotective effects (FIG. 20). Again, LBB-B2 per se did not elicit any neurotoxicity.

Industrial Applicability

In the aforementioned examples, we have shown that both aqueous and basic extracts (LBA and LBB) from Lycium barbarum and the fractionated product from LBA and LBB, LBA-A2, LBB-B1 and LBB-B2 are excellent therapeutic agents for the prevention of neuronal loss by β-amyloid peptide neurotoxicity and in Alzheimer's disease. With the methodology we used for elucidation of molecular mechanisms of neuroprotection, analysis of stress kinases such as JNK and PKR pathway by western-blot analysis have been shown to serve as a technological platform to screen neuroprotective drugs for use in connection with Alzheimer's disease. The methodology we used for isolation and separation of neuroprotective agents demonstrated that the isolation methods can serve as a technological platform to help the screening and manufacturing neuroprotective agents from Chinese and herbal medicine.

References:

The full citation of the references cited above, and incorporated herein by reference are:
Billingsley M L and Kincaid R L (1997) Regulated phosphorylation and dephosphorylation of tau protein: effects on microtubule interaction, intracellular trafficking and neurodegeneration. Biochem. J, 323, 577-591.
Bossy-Wetzel E, Schwarzenbacher R and Lipton S A (2004) Molecular pathways to neurodegeneration. Nature Med., 10, (Suppl.) S2-9.
Chang R C C, Wong A K Y, Ng H K and Hugon J (2002a) Phosphorylation of eukaryotic initiation factor-2α (eIF2α) is associated with neuronal degeneration in Alzheimer's disease. NeuroReport, 13, 2429-2432.
Chang R C C, Suen K C, Ma C H, Elyaman W, Ng H K and Hugon J (2002b) Involvement of doubled-stranded RNA-dependent protein kinase and phosphorylation of eukaryotic initiation factor 2alpha in neuronal degeneration. J Neurochem., 83, 1215-1225.
Deng H B, Cui D P, Jiang J M, Feng Y C, Cai N S and Li D D (2003) Inhibiting effects of Achyranthes Bidentata polysaccharide and Lycium barbarum polysaccharide on nonenzyme glycation in D-galactose induced mouse aging model. Biomed. Enviro. Sci., 16, 267-275.
Engidawork E, Gulesserian T, Seidl R, Cairns N and Lubec G (2001) Expression of apoptosis related proteins in brains of patients with Alzheimer's disease. Neurosci. Lett., 303, 79-82.
Iversen L L, Mortishire-Smith R J, Pollack S J and Shearman M S (1995) The toxicity in vitro of β-amyloid protein. Biochem. J, 311, 1-16.
Kim S Y, Lee E J, Kim H P, Kim Y C, Moon A and Kim Y C (1999) A novel cerebroside from lycii fructurs preserves the hepatic glutathione redox system in primary cultures of rat hepatocytes. Biol. Pharm. Bull., 22, 873-875.
Leroy K, Boutajangout A, Authelet M, Woodgett J R, Anderton B H and Brion J P (2002) The active form of glycogen synthase kinase-3β is associated with granulovacuolar degeneration in neurons in Alzheimer's disease. Acta Neuropathol., 103, 91-99.
Lu X, Xian X, Lu W, Wu X and Gu H. (1999) The regulation of Lycium barbarum on apoptosis of rat spleen in vitro. Zhong Yao Cai; 22, 250-251.
Mandelkow E M and Mandelkow E (1998) Tau in Alzheimer's disease. Trends Cell Biol., 8, 425-427.
Onuki R, Bando Y, uyama E, Katayama T, Kawasaki H., Baba T, Tohyama M and Taira K (2004) An RNA-dependent protein kinase is involved in tunicamycin-induced apoptosis and Alzheimer's disease. EMBO J, 23, 959-968.
Peel A L and Bredesen D E (2003) Activation of the cell stress kinase PKR in Alzheimer's disease and human amyloid precursor protein transgenic mice. Neurobiol. Dis., 14, 52-62.
Rapoport M, Dawson H N, Binder L I, Vitek M P and Ferreira A (2002) Tau is essential to β-amyloid-induced neurotoxicity. Pro. Natl. Acad. Sci. (USA), 99, 6364-6369.
Rohn T T, Head E, Nesse W H, Cotman C W and Cribbs D H (2001) Activation of caspase-8 in the Alzheimer's disease brain. Neuirobiol. Dis., 8, 1006-1016.
Saido T C: Overview-Aβ metabolism: from Alzheimer research to brain aging control. In “Saido T C (ed): Aβ0 metabolism and Alzheimer's disease” pp1-16, (2003).
Savage M J, Lin Y G, Cisllella J R, Flood D G and Scott R W (2002) Activation of c-Jun N-terminal kinase and p38 in an Alzheimer's disease model is associated with amyloid deposition. J Neurosci., 22, 3376-3385.
Scheff S W and Price D A (2003) Synaptic pathology in Alzheimer's disease: a review of ultrastructural studies. Neurobiol. Aging, 24, 1029-1046.
Sindou P, Lesort M, Couratier P, Yardin C, Esclaire F and Hugon J (1994) Glutamate increases tau phosphorylation in primary neuronal cultures from fetal rat cerebral cortex. Brain Res., 646, 124-128.
Stadelmann C, Deckwerth T L, Srinivasan A, Bancher C, Bruck W, Jellinger K and Lassmann H (1999) Activation of caspase-3 in single neurons and autophagic granules of granulovacuolar degeneration in Alzheimer's disease. Evidence for apoptotic cell death. Amer. J Pathol., 155, 1459-1466.
Suen K C, Yu M S, So K F, Chang R C C and Hugon J (2003) The upstream pathway of double-stranded RNA-dependent serine/threonine kinase activation in neurons exposed to β-amyloid peptide neurotoxicity. J Biol. Chem. 278, 49819-49827.
Su J H, Zhao M, Anderson A J, Srinivasan A and Cotman C W (2001) Activated caspase-3 expression in Alzheimer's and aged control brain: correlation with Alzheimer pathology. Brain Res., 898, 350-357.
Wang J, Wang H, Zhang M and Zhang S. (2002) Anti-aging function of polysaccharides from Fructus lycii. Acta Nutrimenta Sinica, 24, 189-194.
Wang Y, Zhao H, Sheng X, Gambino P E, Costello B and Bojanowski K. (2002) Protective effect of Fructus Lycii polysaccharides against time and hyperthermia-induced damage in cultured seminiferous epithelium. J Ethnopharmacol., 82, 169-175.
Xu M, Zhang X and Xu S. (2001) Anti-aging effect of Lycium Chinensis capsule on Drosophila. Journal of Tong Ji University (Med Sci), 22, 143-147.
Yankner B A (1996) Mechanisms of neuronal degeneration in Alzheimer's disease. Neurons, 16, 921-932.
Yu W H, Kumar A, Peterhoff C, Kulnane L S, Uchiyama Y, Lamb B T, Cuervo A M and Nixon R A (2004) Autophagic vacuoles are enriched in amyloid precursor protein-secretase activities: implications for β-amyloid peptide over-production and localization in Alzheimer's disease. Intl. J Biochem. Cell Biol., 36, 2531-2540.
Zhu X, Raina A, Rottkamp C A, Aliev G, Perry G, Boux H and Smith M A (2001) Activation and redistribution of c-Jun N-terminal kinase/stress activated protein kinase in degenerating neurons in Alzheimer's disease. J Neurochem., 76, 435-441.

1. A water soluble polysaccharide-containing extract comprising arabinose, galactose, glucose, xylose, rhamnose, mannose, glucuronic acid and glactouronic acid as analyzed by gas chromatography, exhibiting the ability to inhibit neuronal cell death, and having substantially little or no in vivo toxicity when topically applied to a mammal at a concentration of about 0.1 to 500 μg/ml.

2. An extract according to claim 1 having a molecular mass less than 500 kDa; being substantially insoluble in methanol, ethanol, butanol, acetone and chloroform; containing 15-84% (w/w) carbohydrate, expressed as galuronic and galacturonic acids; and containing up to 93% (w/w) uronic acid, expressed as galuronic and galacturonic acids.

3. An extract according to claim 1 being an extract of a Solanaceae plant.

4. A extract according to claim 1 being an extract of Lycium barbarum.

5. A pharmaceutical formulation comprising an extract according to claim 1 and a pharmaceutically acceptable carrier therefor.

6. A method for producing an extract according to claim 1 comprising soaking a Solanaceae plant material in a lower alcohol, separating the plant material from the alcohol, hot water extracting the material, and subjecting the extract to lower alcohol precipitation.

7. A method for producing an extract according to claim 6, wherein the Solanaceae plant material is a Lycium barbarum material, and the lower alcohol is ethanol.

8. A method for producing an extract according to claim 8 wherein the water has basic pH.

9. A method for inhibiting the Aβ-peptide induced neuronal cell(s) death, said method comprising exposing said cell(s) to an effective amount of an extract according to claim 1

10. A method for attenuation of β-amyloid peptide neurotoxicity in a mammal, said method comprising administering to a mammal an effective amount of an extract according to claim 1.

11. A method according to claim 10 wherein said mammal is human.

12. A method according to claim 11 wherein said extract is administered in combination with a pharmaceutically acceptable carrier.

13. A method for attenuating activation of stress kinase JNK and PKR by Aβ-peptide, said method comprising administering to a mammal an effective amount of an extract according to claim 1.

14. A method for the treatment or prophylaxis of an age-related disorder in a mammal, said method comprising administering an effective amount of an extract according to claim 1 to said mammal.

15. A method of screening a material for neuroprotective activity which comprises conducting a determining the extent of reduction of β-amyloid peptide triggered activation of stress kinase in the presence of said material and determining whether their has been a reduction of β-amyloid peptide triggered activation relative to the activation in the absence of said material.

16. A method of screening a material for neuroprotective activity according to claim 16, wherein the stress kinase is at least one member of the group consisting of c-Jun N-terminal kinase and double-stranded RNA-dependent protein kinase.

17. A method of screening a material for neuroprotective activity according to claim 15, wherein the extent of reduction is determined.
